(19) United States
(12) Patent Application Publication
(10) Pub. No.: US 2026/0207879 A1
(43) Pub. Date: Jul. 23, 2026

(54) APPARATUS FOR CONTROLLING LAMP, CIRCADIAN LAMP AND METHOD FOR CONTROLLING LAMP

(71) Applicant: SAVANT TECHNOLOGIES LLC, East Cleveland, OH (US)

(72) Inventors: Qingkai WANG, Shanghai (CN); Kun XIAO, Shanghai (CN); Carmen PASTORE, Cleveland, OH (US); Laura KICHER, Cleveland, OH (US)

(73) Assignee: SAVANT TECHNOLOGIES LLC, East Cleveland, OH (US)

(21) Appl. No.: 19/441,226

(22) Filed: Jan. 6, 2026

(30) Foreign Application Priority Data
Jan. 23, 2025 (CN) 202510114541.5

(51) Int. Cl. A61M 21/02; A61M 21/00

(52) U.S. Cl. CPC A61M 21/02; A61M 2021/0044; A61M 2205/3561; A61M 2205/80; A61M 2205/82

(57) ABSTRACT

Provided is an apparatus for controlling a lamp, a circadian lamp and a method for controlling a lamp, the apparatus includes a timing module continuously generating timing information when the lamp is turned on and the lamp is turned off, and a lamp control module, receiving the timing information, and controlling, according to the timing information, the lamp to operate in a control mode in which the lamp control module provides a control signal to the lamp on the basis of the timing information, and the lamp adjusts a parameter of the lamp according to the control signal, and the parameter includes at least one of color temperature, color and brightness. The apparatus can achieve a circadian function without the need of network configuration.

### CROSS-REFERENCE TO RELATED APPLICATIONS

This application claims priority to Chinese Patent Application Serial Number 202510114541.5, filed Jan. 23, 2025, which is herein incorporated by reference.

### TECHNICAL FIELD

The present application relates to the technical field of illumination control, and in particular to an apparatus for controlling the color temperature of a lamp, a circadian lamp and a method for controlling the color temperature of a lamp.

### BACKGROUND

On the market, there are already many circadian lamps. The circadian lamps may change illumination depending on time.

However, most current circadian lamps rely on intelligent devices and network configurations, and may require reconfiguration of time each time they are used, and cannot maintain a timing state in a power-off situation, which limits the range of application and convenience thereof.

### SUMMARY OF THE INVENTION

Embodiments of the present application provide an apparatus for controlling a lamp, a circadian lamp, and a method for controlling a lamp, so as to at least solve the technical problem in the prior art that it is difficult to control at least one of color temperature, color, and brightness of a lamp depending on time without an intelligent device and network configuration.

According to embodiments of the present application, an apparatus for controlling a lamp is provided, comprising: a timing module, configured to be capable of continuously generating timing information when the lamp is turned on and the lamp is turned off; and a lamp control module, configured to receive the timing information outputted by the timing module, the lamp control module controlling, according to the timing information, the lamp to operate in a control mode, wherein in the control mode, the lamp control module provides a control signal to the lamp on the basis of the timing information provided by the timing module, and the lamp adjusts a parameter of the lamp according to the control signal, wherein the parameter comprises at least one of color temperature, color and brightness.

In this way, the color temperature of the lamp can be easily adjusted on the basis of time information.

Preferably, the lamp control module comprises: a correspondence data unit, configured to store correspondence data, wherein the correspondence data represents correspondence between the timing information and the parameters.

In this way, how to adjust at least one of color temperature, color and brightness of the lamp can be accurately determined.

Preferably, the timing information represents each time period of a day.

In this way, by differently changing the color temperature of the lamp over each time period of a day, the lamp can adapt to human biological rhythm.

Preferably, the lamp control module further comprises: a control signal generation unit, configured to generate the control signal on the basis of the correspondence data, the control signal causing the lamp to operate at a parameter corresponding to the timing information; and a signal transmission unit, configured to transmit the control signal to the lamp.

In this way, the lamp can be controlled to produce at least one of desired color temperature, color, or brightness at a specific time.

Preferably, the apparatus further comprises: a triggering module, configured to receive an operation command from a user and to control the operation of the timing module on the basis of the operation command.

Preferably, the triggering module receives the operation command from the user, and controls timing start of the timing module or resetting of the timing module on the basis of the operation command.

Preferably, the triggering module is connected to the timing module in a wired or wireless manner, and the triggering module comprises at least one of a wall switch, a remote control unit and a voice control recognition unit.

Preferably, the apparatus further comprises: a power supply module, comprising a main power supply unit and an auxiliary power supply unit, wherein the timing module is capable of being powered continuously by one of the main power supply unit and the auxiliary power supply unit.

Preferably, the power supply module further comprises a switching unit, wherein when the lamp is turned on, the switching unit switches to the main power supply unit, so that the main power supply unit supplies power to the timing module; and when the lamp is turned off, the switching unit switches to the auxiliary power supply unit, so that the auxiliary power supply unit supplies power to the timing module.

Preferably, the main power supply unit is a mains power supplying unit, and the auxiliary power supply unit is a battery power supplying unit.

Preferably, in the control mode, the lamp control module provides the control signal to the lamp on the basis of the timing information provided by the timing module, and the lamp adjusts at least one of color temperature, color, and brightness of the lamp according to the control signal.

According to embodiments of the present application, provided is a circadian lamp, comprising: a light source, the light source being capable of generating light having two or more color temperatures, colors, or brightness; and an apparatus for controlling the light source, the apparatus comprising: a timing module, configured to be capable of continuously generating timing information when the light source is turned on and the light source is turned off; and a lamp control module, configured to receive the timing information outputted by the timing module, the lamp control module controlling, according to the timing information, the light source to operate in a control mode, wherein in the control mode, the lamp control module provides a control signal to the light source on the basis of the timing information provided by the timing module, and the light source adjusts a parameter of the light source according to the control signal, wherein the parameter comprises at least one of color temperature, color and brightness.

Preferably, the lamp control module comprises: a correspondence data unit, configured to store correspondence data, wherein the correspondence data represents correspondence between the timing information and the parameters.

Preferably, the timing information represents each time period of a day.

Preferably, the lamp control module further comprises: a control signal generation unit, configured to generate the control signal on the basis of the correspondence data, the control signal causing the light source to operate at a parameter corresponding to the timing information; and a signal transmission unit, configured to transmit the control signal to the light source.

Preferably, the apparatus further comprises: a triggering module, configured to receive an operation command from a user and to control the operation of the timing module on the basis of the operation command.

Preferably, the triggering module receives the operation command from the user, and controls timing start of the timing module or resetting of the timing module on the basis of the operation command.

Preferably, the triggering module is connected to the timing module in a wired or wireless manner, and the triggering module comprises at least one of a wall switch, a remote control unit and a voice control recognition unit.

Preferably, the apparatus further comprises: a power supply module, comprising a main power supply unit and an auxiliary power supply unit, wherein the timing module is capable of being powered continuously by one of the main power supply unit and the auxiliary power supply unit.

Preferably, the power supply module further comprises a switching unit, wherein when the light source is turned on, the switching unit switches to the main power supply unit, so that the main power supply unit supplies power to the timing module; and when the light source is turned off, the switching unit switches to the auxiliary power supply unit, so that the auxiliary power supply unit supplies power to the timing module.

Preferably, the main power supply unit is a mains power supplying unit, and the auxiliary power supply unit is a battery power supplying unit.

Preferably, in the control mode, the lamp control module provides the control signal to the light source on the basis of the timing information provided by the timing module, and the light source adjusts at least one of color temperature, color, and brightness of the light source according to the control signal.

According to embodiments of the present application, a method for controlling a lamp is provided, comprising: a timing step, which is capable of continuously generating timing information when the lamp is turned on and the lamp is turned off; and a lamp control step of controlling, according to the timing information, the lamp to operate in a control mode, wherein in the control mode, a control signal is provided to the lamp on the basis of the timing information, and the lamp adjusts a parameter of the lamp according to the control signal, wherein the parameter comprises at least one of color temperature, color and brightness.

Preferably, the lamp control step comprises: a correspondence data storage step of storing correspondence data, wherein the correspondence data represents correspondence between the timing information and the parameter.

Preferably, the timing information represents each time period of a day.

Preferably, the lamp control step further comprises: a control signal generation step of generating the control signal on the basis of the correspondence data, the control signal causing the lamp to operate at a parameter corresponding to the timing information; and a signal transmission step of transmitting the control signal to the lamp.

Preferably, before the timing step, the method further comprises: a triggering step of receiving the operation command from the user, and controlling, on the basis of the operation command, the generation of the timing information in the timing step.

Preferably, in the triggering step, the operation command from the user is received, and on the basis of the operation command, the start of generation of the timing information in the timing step or resetting of the timing information is controlled.

Preferably, in the timing step, the timing information can be generated continuously in cases where one of the main power supply unit and the auxiliary power supply unit is used for power supplying.

Preferably, when the lamp is turned on, the timing step can continuously generate the timing information in cases where the main power supply unit supplies power; and when the lamp is turned off, the timing step can continuously generate the timing information in cases where the auxiliary power supply unit supplies power.

Preferably, in the lamp control step, the control signal is provided to the lamp on the basis of the timing information in the timing step, and the lamp adjusts at least one of color temperature, color, and brightness of the lamp according to the control signal.

In embodiments of the present application, a system for controlling a lamp, a circadian lamp, and a method which adjust at least one of color temperature, color and brightness of the lamp by always continuously generating and maintaining timing information are provided, so as to at least solve the technical problem that it is difficult to control at least one of color temperature, color, and brightness of a lamp depending on time without an intelligent device and network configuration; and the product cost and structural complexity are reduced, and the installation environment limitation is eliminated, so that time resetting is easier and more user-friendly.

### BRIEF DESCRIPTION OF THE DRAWINGS

Drawings illustrated herein are used for providing further understanding of the present application and constitute a part of the present application, and the illustrative embodiments of the present application and illustrations thereof are used for explaining the present application, rather than constitute inappropriate limitation on the present application. In the drawings:

FIG. 1 is a schematic diagram of an apparatus for controlling a lamp according to embodiments of the present application;

FIG. 2 is a schematic diagram of a lamp control module according to embodiments of the present application;

FIG. 3 is a schematic diagram of an apparatus for controlling a lamp according to some other embodiments of the present application;

FIG. 4 is a schematic diagram of an apparatus for controlling a lamp according to some other embodiments of the present application;

FIG. 5 is a schematic diagram of a power supply module according to embodiments of the present application;

FIG. 6 is a graph of data stored in a correspondence data unit according to embodiments of the present application;

FIG. 7 is a schematic diagram of a circadian lamp according to embodiments of the present application; and

FIG. 8 is a flowchart of a method for controlling a lamp according to embodiments of the present application.

### DETAILED DESCRIPTION

In order to enable a person skilled in the art to understand the solutions of the present application better, hereinafter, the technical solutions in the embodiments of the present application will be described clearly and thoroughly with reference to the accompanying drawings of embodiments of the present application. Obviously, the embodiments as described are only some of embodiments of the present application, and are not all the embodiments. All other embodiments obtained by a person of ordinary skill in the art on the basis of the embodiments in the present application without involving any inventive effort shall all fall within the scope of protection of the present application.

It should be noted that the terms “first”, “second” etc., in the description, claims, and accompanying drawings of the present application are used to distinguish similar objects, and are not necessarily used to describe a specific sequence or order. It should be understood that the data so used may be interchanged where appropriate so that the embodiments of the present application described herein can be implemented in sequences other than those illustrated or described herein. In addition, terms “comprise” and “have” and any variations thereof are intended to cover a non-exclusive inclusion, for example, a process, method, system, product or device which comprises a series of steps or modules or units is not necessarily limited to those steps or modules or units that are clearly listed, but may comprise other steps or modules or units that are not clearly listed or inherent to these process, method, product, or device.

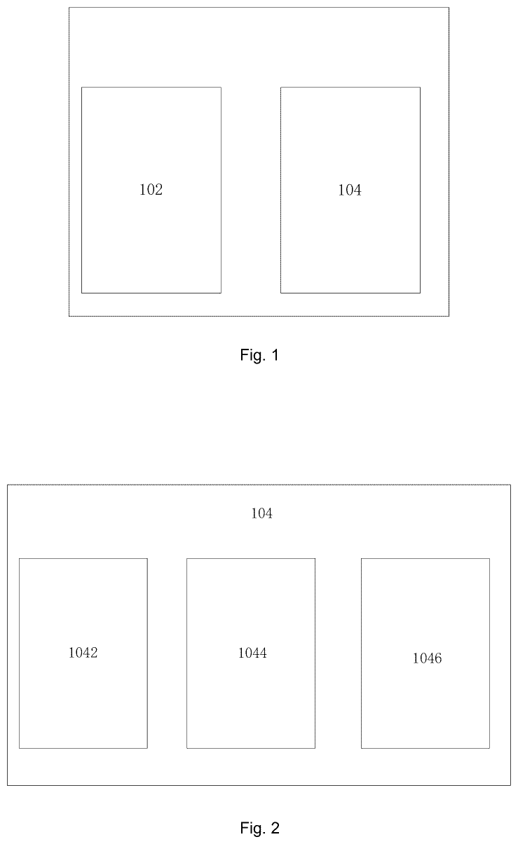

According to embodiments of the present application, an apparatus for controlling a lamp is provided. FIG. 1 is a schematic diagram of an apparatus for controlling a lamp according to embodiments of the present application. As shown in FIG. 1, the apparatus 100 for controlling a lamp comprises: a timing module 102 and a lamp control module 104.

The timing module 102 is configured to be capable of continuously generating timing information when the lamp is turned on and the lamp is turned off. In other words, the timing module 102 can continuously generate the timing information no matter whether an external power supply of the lamp supplies power to the lamp. The lamp control module 104 is configured to receive the timing information outputted by the timing module 102, and the lamp control module 104 controls, according to the timing information, the lamp to operate in a control mode, wherein in the control mode, the lamp control module 104 provides a control signal to the lamp on the basis of the timing information provided by the timing module 102, and the lamp adjusts a parameter of the lamp according to the control signal, wherein the parameter comprises at least one of color temperature, color and brightness of light emitted by the lamp. In this way, the color temperature, color and brightness of the lamp can be easily adjusted on the basis of time information.

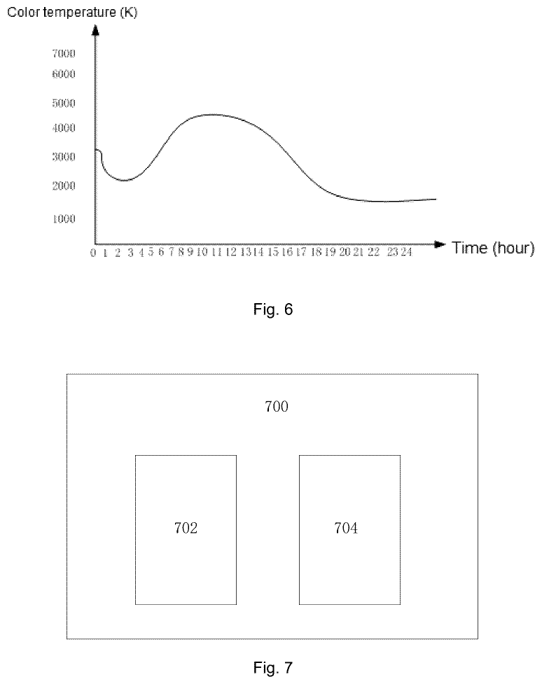

FIG. 2 is a schematic diagram of the lamp control module according to embodiments of the present application. As shown in FIG. 2, the lamp control module 104 comprises: a correspondence data unit 1042, a control signal generation unit 1044, and a signal transmission unit 1046. The correspondence data unit 1042 is configured to store correspondence data, wherein the correspondence data represents correspondence between the timing information and the parameter of the lamp (e.g. at least one of color temperature, color and brightness of the light emitted by the lamp). For example, the timing information may represent each time period of a day. The correspondence data stored by the correspondence data unit 1042 has, for example, a graph of data as shown in FIG. 6. In the graph of data shown in FIG. 6, a horizontal axis can represent various time periods within a day (i.e. various time periods within 24 hours of a day, e.g. 0th, 1st, 2nd, 3rd, 4th, 5th, 6th, 7th, 8th, 9th, 10th, 11th, 12th, 13th, 14th, 15th, 16th, 17th, 18th, 19th, 20th, 21st, 22nd, 23rd, and 24th hour), and a vertical axis can represent the color temperature of the lamp. For example, at 0th hour, light emitted by the lamp has a first color temperature; at 1st hour, light emitted by the lamp has a second color temperature; at 3rd hour, light emitted by the lamp has a third color temperature, and so on. Thus, the correspondence data stored by the correspondence data unit 1042 represents the corresponding relationship between each time period of a day and the color temperature of light emitted by the lamp. Therefore, on the basis of the timing information, the color temperature corresponding to the timing information can be inferred. Although the vertical axis in FIG. 6 represents the color temperature of the lamp, the vertical axis in FIG. 6 may also represent at least one of color and brightness of the lamp. The control signal generation unit 1044 is configured to generate the control signal on the basis of the timing information and the correspondence data, the control signal causing the lamp to operate at a parameter (e.g. color temperature) corresponding to the timing information. The signal transmission unit 1046 is configured to transmit the control signal to the lamp. In this way, by differently changing the color temperature, color and brightness of the light emitted by the lamp over each time period of a day, the lamp can adapt to human biological rhythm.

The control signal generation unit 1044 is configured to obtain color temperature, color or brightness corresponding to the timing information on the basis of the timing information outputted by the timing module 102 and with reference to the correspondence data stored in the correspondence data unit 1042, and then to generate the control signal, wherein the control signal causes the lamp to operate according to the color temperature, color or brightness corresponding to the timing information.

Next, the signal transmission unit 1046 transmits to the lamp the control signal generated by the control signal generation unit 1044, so that the lamp, when turned on, can emit light with corresponding color temperature, color, or brightness.

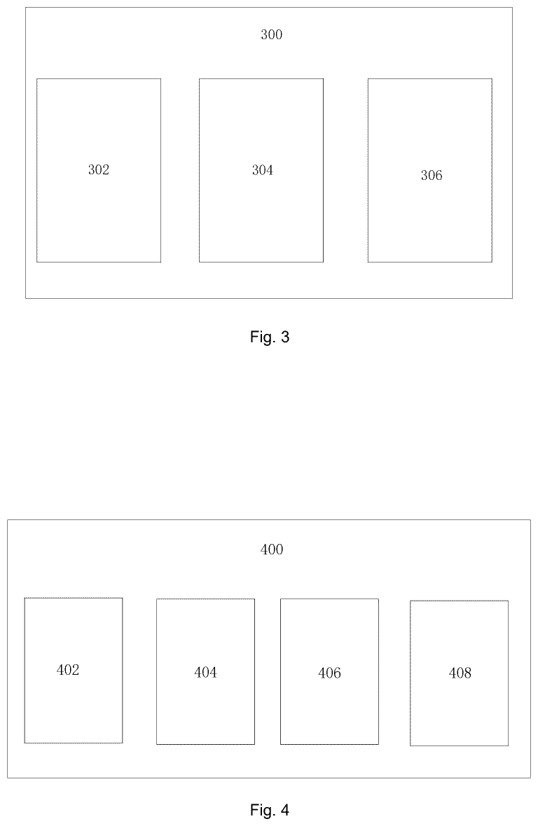

FIG. 3 is a schematic diagram of an apparatus for controlling a lamp according to some other embodiments of the present application. As shown in FIG. 3, in addition to a timing module 302 and a lamp control module 304, the apparatus 300 for controlling a lamp according to some other embodiments of the present application further comprises a triggering module 306. The triggering module 306 is configured to receive an operation command from a user and to control the operation of the timing module 302 on the basis of the operation command. For example, the triggering module 306 is connected to the timing module 302 in a wired (e.g. physical wiring) or wireless (e.g. by Bluetooth) manner. The triggering module 306 can be implemented in the form of at least one of a wall switch, a remote controller, and a voice control recognition unit. The triggering module 306 receives the operation command from the user (for example, in cases where the triggering module 306 is implemented in the form of a wall switch, the triggering module 306 may take a behavior that the user quickly turns on and off the wall switch twice or more times as the operation command of the user; in cases where the triggering module 306 is implemented in the form of a remote controller, the triggering module 306 may take a behavior that the user operates a specific button on the remote controller as the operation command of the user; and in cases where the triggering module 306 is implemented in the form of a voice control recognition unit, the triggering module 306 may take a sentence spoken by the user which comprises a specific word as the operation command of the user), and the triggering module controls, on the basis of the operation command, timing start of the timing module 302 or resetting of the timing module 302 to make the timing information thereof return to zero. Therefore, without the need of network configuration, and without the need of operating a specific application program on an intelligent device, the user can easily reset the timing module or start timing of the timing module, so that each time the lamp is turned on, according to a set plan, the lamp can display at least one of different color temperatures, colors and brightness according to different times, thereby realizing a circadian function.

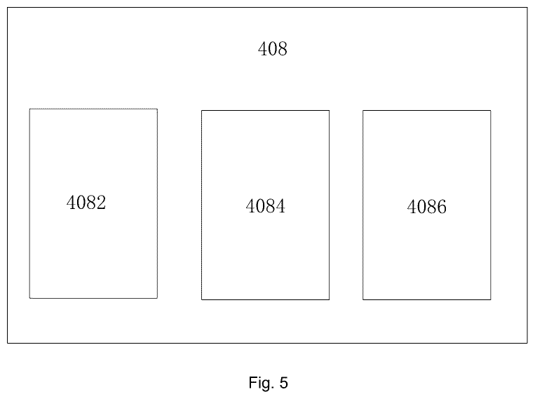

FIG. 4 is a schematic diagram of an apparatus for controlling a lamp according to some other embodiments of the present application. As shown in FIG. 4, in addition to a timing module 402, a lamp control module 404, and a triggering module 406, the apparatus 400 for controlling a lamp according to some other embodiments of the present application further comprises a power supply module 408. As shown in FIG. 5, the power supply module 408 comprises a main power supply unit 4082, an auxiliary power supply unit 4084, and a switching unit 4086. When the lamp is turned on, the switching unit 4086 switches to the main power supply unit 4082, so that the main power supply unit 4082 supplies power to the timing module 402; and when the lamp is turned off, the switching unit 4086 switches to the auxiliary power supply unit 4084, so that the auxiliary power supply unit 4084 supplies power to the timing module 402. For example, the main power supply unit 4082 may be a mains power supply unit, and the auxiliary power supply unit 4084 may be a battery power supply unit. When the lamp is turned on, in addition to supplying power to the timing module 402, the main power supply unit 4082 may also charge the auxiliary power supply unit 4084. Thus, by making the power supply module 408 comprise the main power supply unit 4082, the auxiliary power supply unit 4084, and the switching unit 4086, the timing module can also be powered uninterruptedly when the lamp is turned off (that is, the main power supply unit 4082 does not supply power to the lamp), thereby continuously generating the timing information. Even when the lamp is turned off, the timing module can also continue to work; therefore, each time when the lamp is turned on, without resetting the timing module, the lamp can display, according to a set plan, at least one of different color temperatures, colors and brightness according to different times, thereby realizing a circadian function. In addition, the auxiliary power supply unit 4084 may be disposed at a position that is easily touched by the user, so that the timing module may be reset by simply removing the auxiliary power supply unit 4084. Furthermore, since the power supply module 408 comprises the main power supply unit 4082, the auxiliary power supply unit 4084 and the switching unit 4086, switching between a normal operation mode and a low power consumption mode is achieved according to the power supply mode of the lamp, and further low power consumption of an illumination device and a control apparatus therefor is ensured while achieving the circadian function of the lamp.

FIG. 7 is a schematic diagram of a circadian lamp according to embodiments of the present application. As shown in FIG. 7, the circadian lamp 700 comprises: a light source 702, capable of generating light having two or more color temperatures, colors, or brightness; and an apparatus 704 for controlling the light source. The light source 702 may be an LED illumination apparatus. In addition, the apparatus 704 for controlling the light source may for example have the configurations as shown in FIGS. 1, 3 and 4.

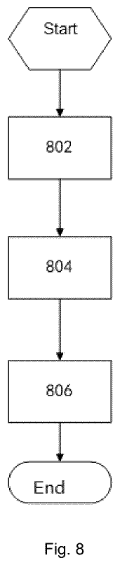

FIG. 8 is a flowchart of a method for controlling a lamp according to embodiments of the present application. As shown in FIG. 8, the method for controlling a lamp comprises the following steps: a triggering step 802 of receiving an operation command from a user, and controlling the generation of timing information in a timing step 804 on the basis of the operation command; the timing step 804, which is capable of continuously generating the timing information when the lamp is turned on and the lamp is turned off; and a lamp control step 806 of controlling, according to the timing information, the light to operate in a control mode, wherein in the control mode, a control signal is provided to the lamp on the basis of the timing information, and the lamp adjusts a parameter of the lamp according to the control signal, wherein the parameter comprises at least one of color temperature, color and brightness. In addition, the lamp control step 806 comprises: a correspondence data storage step of storing correspondence data, wherein the correspondence data represents correspondence between the timing information and the parameter. a control signal generation step of generating the control signal on the basis of the correspondence data and the timing information, the control signal causing the lamp to operate at a parameter corresponding to the timing information; and a signal transmission step of transmitting the control signal to the lamp.

In accordance with exemplary embodiments of the present application, although only control of at least one of color temperature, color, and brightness of the lamp depending on time is described, other parameters of the lamp can also be controlled depending on time, as can be appreciated by a person skilled in the art.

Advantages of the solution lie in: by intelligent adjustment of color temperature, brightness and color, the illumination effect can be automatically optimized according to time change, thereby improving the quality of life and working efficiency of the user. In addition, the power supply switching function provided thereby ensures the continuity of timing and control functions in the case of power outage, and provides a more stable and reliable illumiation experience for the user. In addition, diversified user operation manners, such as wall switch, remote controller and voice control, make the control more convenient, satisfy requirements in different scenarios, and improve the practicality and user friendliness of the apparatus. Such a solution is not only applicable to homes and offices, but also applicable to various public places, such as schools, hospitals and shopping malls, and can provide the best illumination experience according to different environments and requirements. Moreover, the intelligent design thereof also enables the device to operate stably for a long time, reduces the maintenance costs, and increases the use efficiency.

In the described embodiments of the present application, the illustration of each embodiment has its own emphasis. For the part not detailed in a certain embodiment, please refer to the relevant illustration in other embodiments.

In the several embodiments provided in the present application, it should be understood that the disclosed technical content may be implemented in other manners. The apparatus embodiments described above are merely illustrative. For example, the division of a unit or a module is only a logical function division, and in actual implementation, there may be another division manner, for example, multiple units or modules or assemblies may be combined, or can be integrated into another system, or some features can be ignored or not executed. In addition, the coupling or direct coupling or communication connection shown or discussed may be an indirect coupling or communication connection through some interfaces, modules or units, and may be electrical or other forms.

The units or modules described as separate components may or may not be physically separated, and the components displayed as units or modules may or may not be physical units or modules, that is, may be located in one place, or may be distributed in a plurality of network units or modules. Some or all of the units or modules may be selected according to actual needs to achieve the purpose of the solutions of the embodiments.

In addition, functional units or modules in the embodiments of the present application may be integrated into one processing unit or module, or the units or modules may exist alone physically, or two or more units or modules are integrated into one unit or module. The described integrated unit or module can be embodied in the form of hardware or in the form of a software functional unit or module.

If the integrated unit is implemented in the form of a software functional unit and is sold or used as an independent product, the integrated unit can be stored in a computer-readable storage medium. On the basis of such understanding, the part of the technical solution of the present application that contributes in essence or to the prior art or all or part of the technical solution may be embodied in the form of a software product stored in a storage medium. Several instructions are included in the storage medium to enable a computer device (which may be a personal computer, server or network device, etc.) to execute all or some of the steps of the methods of various embodiments of the present application. Moreover, the storage medium above comprises: media such as a USB flash disk, a Read-Only Memory (ROM), a Random Access Memory (RAM), a removable hard disk, a magnetic disk, or an optical disk which can store program codes.

The illustration above only relates to preferred embodiments of the present application. It should be noted that for a person of ordinary skill in the present technical field, several improvements and modifications can also be made without departing from the principle of the present application, and these improvements and modifications shall also be considered as within the scope of protection of the present application.

1. An apparatus for controlling a lamp, the apparatus comprising:
a timing module, configured to be capable of continuously generating timing information when the lamp is turned on and the lamp is turned off; and
a lamp control module, configured to receive the timing information outputted by the timing module, the lamp control module controlling, according to the timing information, the lamp to operate in a control mode, wherein in the control mode, the lamp control module provides a control signal to the lamp on the basis of the timing information provided by the timing module, and the lamp adjusts a parameter of the lamp according to the control signal, wherein the parameter comprises at least one of color temperature, color and brightness.

2. The apparatus for controlling a lamp according to claim 1, wherein the lamp control module comprises:
a correspondence data unit, configured to store correspondence data, wherein the correspondence data represents correspondence between the timing information and the parameter.

3. The apparatus for controlling a lamp according to claim 1, wherein the timing information represents each time period of a day.

4. The apparatus for controlling a lamp according to claim 2, wherein the lamp control module further comprises:
a control signal generation unit, configured to generate the control signal on the basis of the correspondence data and the timing information, the control signal causing the lamp to operate at a parameter corresponding to the timing information; and
a signal transmission unit, configured to transmit the control signal to the lamp.

5. The apparatus for controlling a lamp according to claim 1, wherein the apparatus further comprises:
a triggering module, configured to receive an operation command from a user and to control the operation of the timing module on the basis of the operation command.

6. The apparatus for controlling a lamp according to claim 5, wherein the triggering module receives the operation command from the user, and controls timing start of the timing module or resetting of the timing module on the basis of the operation command.

7. The apparatus for controlling a lamp according to claim 5, wherein the triggering module is connected to the timing module in a wired or wireless manner, and the triggering module comprises at least one of a wall switch, a remote control unit and a voice control recognition unit.

8. The apparatus for controlling a lamp according to claim 1, wherein the apparatus further comprises:
a power supply module, comprising a main power supply unit and an auxiliary power supply unit, wherein the timing module is capable of being powered continuously by one of the main power supply unit and the auxiliary power supply unit.

9. The apparatus for controlling a lamp according to claim 8, wherein the power supply module further comprises a switching unit, wherein when the lamp is turned on, the switching unit switches to the main power supply unit, so that the main power supply unit supplies power to the timing module; and when the lamp is turned off, the switching unit switches to the auxiliary power supply unit, so that the auxiliary power supply unit supplies power to the timing module.

10. The apparatus for controlling a lamp according to claim 8, wherein the main power supply unit is a mains power supplying unit, and the auxiliary power supply unit is a battery power supplying unit.

11. The apparatus for controlling a lamp according to claim 1, wherein in the control mode, the lamp control module provides the control signal to the lamp on the basis of the timing information provided by the timing module, and the lamp adjusts at least one of color temperature, color, and brightness of the lamp according to the control signal.

12. A circadian lamp, comprising:
a light source, the light source being capable of generating light having two or more color temperatures, colors, or brightness; and
an apparatus for controlling the light source, the apparatus comprising:
a timing module, configured to continuously generate timing information when the light source is turned on and the light source is turned off; and
a lamp control module, configured to receive the timing information outputted by the timing module, the lamp control module controlling, according to the timing information, the light source to operate in a control mode, wherein in the control mode, the lamp control module provides a control signal to the light source on the basis of the timing information provided by the timing module, and the light source adjusts a parameter of the light source according to the control signal, wherein the parameter comprises at least one of color temperature, color and brightness.

13. The circadian lamp according to claim 12, wherein the lamp control module comprises:
a correspondence data unit, configured to store correspondence data, wherein the correspondence data represents correspondence between the timing information and the parameter.

14. The circadian lamp according to claim 12, wherein the timing information represents each time period of a day.

15. The circadian lamp according to claim 13, wherein the lamp control module further comprises:
a control signal generation unit, configured to generate the control signal on the basis of the correspondence data and the timing information, the control signal causing the light source to operate at a parameter corresponding to the timing information; and
a signal transmission unit, configured to transmit the control signal to the light source.

16. The circadian lamp according to claim 12, wherein the apparatus further comprises:
a triggering module, configured to receive an operation command from a user and to control the operation of the timing module on the basis of the operation command.

17. The circadian lamp according to claim 16, wherein the triggering module receives the operation command from the user, and controls timing start of the timing module or resetting of the timing module on the basis of the operation command.

18. The circadian lamp according to claim 16, wherein the triggering module is connected to the timing module in a wired or wireless manner, and the triggering module comprises at least one of a wall switch, a remote control unit and a voice control recognition unit.

19. The circadian lamp according to claim 12, wherein the apparatus further comprises:
a power supply module, comprising a main power supply unit and an auxiliary power supply unit, wherein the timing module is capable of being powered continuously by one of the main power supply unit and the auxiliary power supply unit.

20. The circadian lamp according to claim 19, wherein the power supply module further comprises a switching unit, wherein when the light source is turned on, the switching unit switches to the main power supply unit, so that the main power supply unit supplies power to the timing module; and when the light source is turned off, the switching unit switches to the auxiliary power supply unit, so that the auxiliary power supply unit supplies power to the timing module.

21. The circadian lamp according to claim 19, wherein the main power supply unit is a mains power supplying unit, and the auxiliary power supply unit is a battery power supplying unit.

22. The circadian lamp according to claim 12, wherein in the control mode, the lamp control module provides the control signal to the light source on the basis of the timing information provided by the timing module, and the light source adjusts at least one of color temperature, color, and brightness of the light source according to the control signal.

23. A method for controlling a lamp, the method comprising:
continuously generating timing information when the lamp is turned on and the lamp is turned off; and
controlling, according to the timing information, the lamp to operate in a control mode, wherein in the control mode, a control signal is provided to the lamp on the basis of the timing information, and the lamp adjusts a parameter of the lamp according to the control signal, wherein the parameter comprises at least one of color temperature, color and brightness.

24. The method for controlling a lamp according to claim 23, wherein the lamp control step comprises:
storing correspondence data, wherein the correspondence data represents correspondence between the timing information and the parameter.

25. The method for controlling a lamp according to claim 23, wherein the timing information represents each time period of a day.

26. The method for controlling a lamp according to claim 24, wherein the lamp control step further comprises:
generating the control signal on the basis of the correspondence data and the timing information, the control signal causing the lamp to operate at a parameter corresponding to the timing information; and
transmitting the control signal to the lamp.

27. The method for controlling a lamp according to claim 23, wherein before continuously generating timing information, the method further comprises:
receiving an operation command from a user, and controlling the generation of the timing information on the basis of the operation command.

28. The method for controlling a lamp according to claim 27, wherein the operation command from the user is received, and on the basis of the operation command, the start of generation of the timing information or resetting of the timing information is controlled.

29. The method for controlling a lamp according to claim 27, wherein the timing information can be generated continuously when one of the main power supply unit and the auxiliary power supply unit is used for power supplying.

30. The method for controlling a lamp according to claim 29, wherein when the lamp is turned on, continuously generate the timing information in cases where the main power supply unit supplies power; and when the lamp is turned off, continuously generate the timing information in cases where the auxiliary power supply unit supplies power.

31. The method for controlling a lamp according to claim 23, wherein when controlling the lamp in control mode, the control signal is provided to the lamp on the basis of the timing information, and the lamp adjusts at least one of color temperature, color, and brightness of the lamp according to the control signal.
